(19) United States
(12) Patent Application Publication
(10) Pub. No.: US 2019/0051639 A1
(43) Pub. Date: Feb. 14, 2019

(54) STICKER ELECTRONICS

(71) Applicant: KING ABDULLAH UNIVERSITY OF SCIENCE AND TECHNOLOGY, Thuwal (SA)

(72) Inventors: Muhammad Mustafa HUSSAIN, Austin, TX (US); Galo Andrés TORRES SEVILLA, Tungurahua (EC); Marlon Steven DIAZ CORDERO, Thuwal (SA)

(21) Appl. No.: 16/078,098

(22) PCT Filed: Feb. 28, 2017
(86) PCT No.: PCT/IB2017/051170

§ 371 Date: Aug. 21, 2018

Related U.S. Application Data
(60) Provisional application No. 62/301,611, filed on Feb. 29, 2016.

(51) Int. Cl. H01L 25/16; H05K 1/18; H01L 23/538; H05K 1/16; H01L 33/62; H05K 1/03; H05K 3/46; H01L 49/02; H01F 38/14; G01N 33/00; G01W 1/14

(52) U.S. Cl. CPC H01L 25/162; H02J 7/025; H01L 23/5383; H05K 1/165; H01L 33/62; H05K 1/0386; H01L 23/5387; H01L 23/5389; H05K 3/4611; H01L 25/167; H01L 28/10; H01F 38/14; G01N 33/004; G01W 1/14; H05K 2201/10037; H05K 2201/10151; H05K 2201/10106; H01L 2933/0033; H05K 1/189

(57) ABSTRACT

Electronic stickers may be manufactured on flexible substrates as layers and packaged together. The package may then have an adhesive applied to one side to provide capability for sticking the electronic devices to surfaces. The stickers can be wrappable, placed on surfaces, glued on walls or mirrors or wood or stone, and have electronics which may or may not be ultrathin. Packaging for the electronic sticker can use polymer on cellulose manufacturing and/or three dimensional (3-D) printing. The electronic stickers may provide lighting capability, sensing capability, and/or recharging capabilities.

### CROSS-REFERENCE TO RELATED PATENT APPLICATIONS

This application claims priority and benefit from U.S. Provisional Patent Application No. 62/301,611, filed Feb. 29, 2016, for “STICKER ELECTRONICS,” the entire contents of which are incorporated in their entirety herein by reference.

### FIELD OF THE DISCLOSURE

The instant disclosure relates to electronic devices. More specifically, portions of this disclosure relate to small electronic devices packaged as a sticker.

### BACKGROUND

Electronic devices are continually shrinking in size and providing new capabilities with smaller footprints. For example, televisions (TVs) have evolved from tube-based black-and-white devices of several feet in thickness to full-color high-definition displays that are only fractions of an inch in thickness. In any device or apparatus that does not have storage space as part of its functionality, there is generally little reason to not further reduce the size of that device or apparatus. Conventional semiconductor manufacturing has worked to reduce the size of many devices. However, conventional semiconductor manufacturing technology is not well suited to all kinds of devices, in part because of the need for a rigid substrate, such as a silicon wafer.

### SUMMARY

Flexible substrates can be much easier for constructing consumer device and provide more desirable characteristics and reduced cost. Some techniques for manufacturing flexible semiconductor substrates are described in U.S. Pat. No. 9,209,083 to Hussain et al. and entitled “Integrated Circuit Manufacturing for Low-Profile and Flexible Devices” and U.S. Patent Application Publication No. 2014/0239459 to Hussain et al. and entitled “Method for Producing Mechanically Flexible Silicon Substrate,” both of which are incorporated by reference in their entirety. These techniques are example techniques that may be employed in manufacturing the devices, such as the electronic stickers, described below. Other techniques may also be suitable for manufacturing these devices.

Layers of devices may be stacked on each other on a flexible substrate to form a flexible device, such as an electronic sticker. Different functionality can be incorporated onto the different layers and packaged together as a low-cost electronic device, which may be used as in an Internet of Things (IoT) network. Some example functionality for the electronic stickers includes lighting, control, power charging, and environmental sensing. Further, functionality can be combined, such as to provide for controllable lighting in a single electronic sticker.

According to one embodiment, an apparatus may include a flexible substrate comprising a base for one or more electronic devices and/or one or more layers on the flexible substrate, wherein the one or more layers form components for the one or more electronic devices. In some embodiments, a top layer of the one or more layers is exposed to an environment around the apparatus and includes components to interact with the environment.

In certain embodiments, at least one layer of the one or more layers may include an inductive power coil configured to receive power for operating the one or more electronic devices; at least one layer of the one or more layers may include one or more light emitting diodes (LEDs); the light emitting diodes (LEDs) may be configured to receive power from the inductive power coil; at least one layer of the one or more layers may include a wireless module; the wireless module may be coupled to the inductive power coil and to the light emitting diodes (LEDs); the wireless module may be configured to receive commands to operate the light emitting didoes (LEDs) and to operate the light emitting diodes (LEDs) based, at least in part, on the received commands; the apparatus may include an adhesive backing on the flexible substrate to form a sticker; the apparatus may also include a flexible battery coupled to the one or more layers; the flexible substrate may be packaged using polymer on cellulose manufacturing using 3D printing; at least one layer of the one or more layers may include one or more sensors; the one or more sensors may be configured to receive power from the inductive power coil; at least one layer of the one or more layers may include a wireless module; the wireless module may be coupled to the inductive power coil and to the one or more sensors; the wireless module may be configured to receive data from the one or more sensors and to wirelessly communicate the received data to a client device; the one or more sensors may include at least one of a smoke detector and a carbon monoxide detector; and/or the one or more sensors may include a rain gauge.

The foregoing has outlined rather broadly certain features and technical advantages of embodiments of the present invention in order that the detailed description that follows may be better understood. Additional features and advantages will be described hereinafter that form the subject of the claims of the invention. It should be appreciated by those having ordinary skill in the art that the conception and specific embodiment disclosed may be readily utilized as a basis for modifying or designing other structures for carrying out the same or similar purposes. It should also be realized by those having ordinary skill in the art that such equivalent constructions do not depart from the spirit and scope of the invention as set forth in the appended claims. Additional features will be better understood from the following description when considered in connection with the accompanying figures. It is to be expressly understood, however, that each of the figures is provided for the purpose of illustration and description only and is not intended to limit the present invention.

### BRIEF DESCRIPTION OF THE DRAWINGS

For a more complete understanding of the disclosed system and methods, reference is now made to the following descriptions taken in conjunction with the accompanying drawings.

FIG. 1 is a top view of an electronic sticker with light emitting diodes (LEDs) according to one embodiment of the disclosure.

FIG. 2 is side view of the electronic sticker with LEDs of FIG. 1.

FIG. 3 is a partially exploded perspective view of the electronic sticker with LEDs of FIG. 1.

FIG. 4 is a flowchart of a method of making an electronic sticker according to one embodiment of the disclosure.

### DETAILED DESCRIPTION

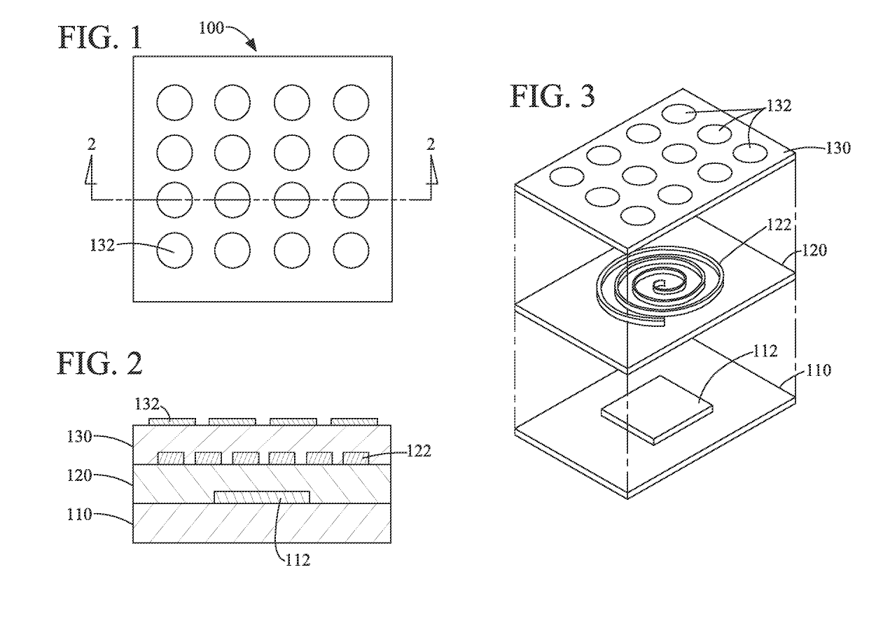

Electronic stickers may be manufactured on flexible substrates as layers and packaged together. The package may then have an adhesive applied to one side to provide capability for sticking the electronic devices to surfaces. The stickers can be wrappable, placed on surfaces, glued on walls or mirrors or wood or stone, and have electronics which may or may not be ultrathin. Packaging for the electronic sticker can use polymer on cellulose manufacturing and/or three dimensional (3-D) printing. In one example, the electronic stickers are used to provide lighting capability as shown in FIGS. 1-3. FIG. 1 is a top view of an electronic sticker 100 with light emitting diodes (LEDs) according to one embodiment of the disclosure. FIG. 2 is side view of the electronic sticker 100 with LEDs of FIG. 1. FIG. 3 is a partially exploded perspective view of the electronic sticker 100 with LEDs of FIG. 1. An electronic sticker 100 may include layers 110, 120, and 130. Although three layers are shown in FIG. 1, an electronic sticker may include more or less layers.

The first layer 110 may include a wireless module 112, such as an integrated circuit (IC) including a controller or processor and associated radio circuitry for performing wireless communications. The wireless module 112 may provide support for wireless communications according to IEEE 802.11 WiFi, Bluetooth, Long Term Evolution (LTE), and/or other wireless communication standards.

The second layer 120 may include a power coil 122 for receiving energy to power electronic components on the layers 110, 120, and/or 130. For example, the power coil 122 may be inductively coupled to a permanent or mobile power source to energize the system 100. In one example, the system 100 may be applied as a sticker to a receiving element that is affixed to an infrastructure power system, such as the electrical system in a home. In another example, the system 100 may be applied as a sticker at a location and a mobile power unit or a mobile phone brought into proximity with the system 100 to temporarily power the system 100. In one embodiment, a flexible battery (not shown) may be integrated with the system 100 or an external battery (not shown) may be attached to the system 100. The battery may be coupled to the power coil 122 to receive energy to be stored. The system 100 can then be operated in the absence of a power source for short or long durations of time based on a capacity of the battery. Although a battery is described, other configurations may be integrated with and attached to the system 100 for energy storage. For example, metal-insulator-metal (MIM) capacitors (not shown) may be manufactured in a layer of the system 100 and used to store energy received from the coil 122.

The third layer 130 may include light emitting diodes (LEDs) 132 that convert energy to visible light to illuminate areas. The LEDs 132 may be coupled to other layers, such as to the power coil 122 on the second layer 120 and to the wireless module 112 on the first layer 110. The LEDs 132 may receive energy from the power coil 122 under control of the wireless module 112. For example, a switch (not shown) may couple the LEDs 132 to the power coil 122, and the switch may be toggled on and off by the wireless module 112 to turn on and off the LEDs 132. As another example, dimmer circuitry (not shown) may be coupled between the LEDs 132 and the power coil 122 and controlled by the wireless module 112 to control an intensity of the light output of the LEDs 132.

The wireless module 112 may be controlled from a local device, such as a user's mobile phone located in the room with the LEDs 132. In one scenario, a user may tap their mobile phone to the electronic sticker, upon which the power coil 122 is energized by the battery in the mobile phone to activate the wireless module 112. The user's mobile phone then pairs with the wireless module 112, upon which a control application activates on the user's mobile phone. The control application may include a power button for turning on and off the LEDs 132, a dimmer slider for controlling an intensity of the LEDs 132, and/or a color selector for selecting a color of light emitted by the LEDs 132. The wireless module 112 may also or alternatively be controlled from a remote device, such as a user's laptop while the user is away from their home.

Although a light emitting electronic sticker is described above, other functionality may be provided in the various layers of the system 100. In another embodiment, an electronic sticker may be configured as a smoke detector. Smoke sensors may be integrated in a third layer 130 that is outward facing towards the environment around the sticker. The smoke sensors may be coupled to a wireless module that can communicate information from the smoke sensors, such as the presence of smoke in the area. Further, although smoke sensors are described, any sensor may be integrated in the electronic sticker. For example, a carbon monoxide detector may be included with or in the alternate to the smoke sensor. As another example, a water sensor may be integrated with an electronic sticker and used to detect leaky pipes or overflowing tubs and sinks. In yet another example, a temperature sensor or ultraviolet (UV) light sensor may be integrated in an electronic sticker. The sticker may be placed in rooms throughout a house, on windows throughout the house, and/or on a patio or in the yard. A user may activate the sensors with their mobile phone to obtain measurements from the sensors integrated in the stickers. For example, a user may tap their phone against the electronic sticker to energize the power coil and pair with the wireless module, which then transmits to the user's phone a measurement from the integrated sensor, such as the local temperature. The user's mobile device may then store the measurement for later viewing or automatically launch an application that provides the information to the user. In another example, a rain gauge sensor may be integrated in an electronic sticker. The rain gauge may be used to determine recent rainfall amounts and the wireless module activated to send commands to a sprinkler system.

In yet another embodiment, the electronic stickers may provide services to other devices. For example, an electronic sticker may provide recharging capability to mobile devices. Such an electronic sticker may include the power coil 122 shown in FIG. 1, but use the power coil 122 to transmit energy to other devices. The electronic sticker may be hard-wired to an electrical system, such as a house's 120V AC electrical system. One layer of the electronic sticker may include appropriate circuitry to generate an appropriate level signal for the power coil 122. When a user's mobile device is placed against the sticker, the user's mobile device may receive energy to recharge its battery. Although similar charging pads are conventionally available, they all involve rigid structures that are not suitable in all areas and that are bulky and unsightly. When manufactured as an electronic sticker, such a charging device may be ultrathin, conform to surface shapes, and adhere to the surface in a much smaller package than conventional wireless charging pads.

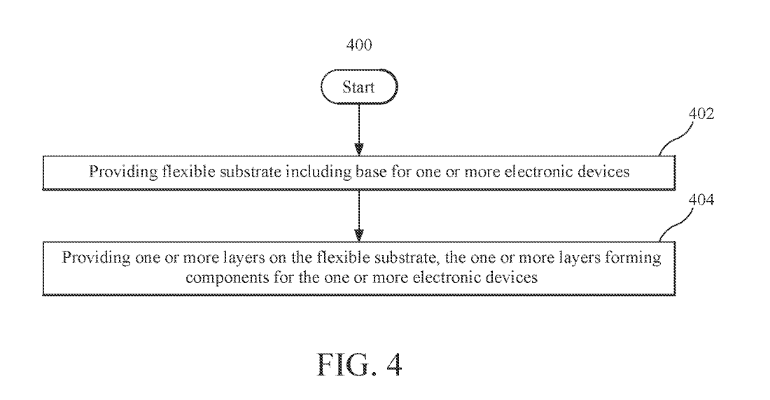

FIG. 4 is a flowchart of a method 400 of making an electronic sticker according to one embodiment of the disclosure. In operation 402, a flexible substrate comprising a base for one or more electronic devices may be provided. For example, first layer 110 may form the flexible substrate for the one or more electronic devices. In operation 404, one or more layers may be provided on the flexible substrate. The one or more layers may form components for the one or more electronic devices. A top layer of the one or more layers may be exposed to an environment around the top layer and include components to interact with the environment. For example, third layer 130 may form the top layer of the one or more layers.

At least one layer of the one or more layers may include an inductive power coil configured to receive power for operating the one or more electronic devices. For example, second layer 120 may include power coil 122 for receiving energy to power electronic components on the layers 110, 120, and/or 130. At least one layer of the one or more layers may include one or more light emitting diodes (LEDs). The LEDs may be configured to receive power from the inductive power coil. For example, the third layer 130 may include LEDs 132 that may receive energy from the power coil 122. At least one layer of the one or more layers may include a wireless module. The wireless module may be coupled to the inductive power coil and to the LEDs. For example, the LEDs may receive energy from the power coil 122 under control of the wireless module 112. The wireless module may be configured to receive commands to operate the LEDs and to operate the light emitting diodes LEDs based, at least in part, on the received commands.

The method 400 may further include providing an adhesive backing on the flexible substrate to form a sticker. The method 400 may further include coupling a flexible battery to the one or more layers. The method 400 may further include packaging the flexible substrate using polymer on cellulose manufacturing using 3D printing.

At least one layer of the one or more layers may include one or more sensors. The one or more sensors may be configured to receive power from the inductive power coil. At least one layer of the one or more layers may include a wireless module coupled to the inductive power coil and to the one or more sensors. The wireless module may be configured to receive data from the one or more sensors and to wirelessly communicate the received data to a client device. The one or more sensors may include at least one of a smoke detector and a carbon monoxide detector. For example, third layer 130 may include a smoke sensor integrated therein. The one or more sensors may include a rain gauge.

Although the present disclosure and certain representative advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

What is claimed is:

1. An apparatus, comprising:
a flexible substrate comprising a base for one or more electronic devices; and
one or more layers on the flexible substrate, wherein the one or more layers form components for the one or more electronic devices,
wherein a top layer of the one or more layers is exposed to an environment around the apparatus and includes components to interact with the environment.

2. The apparatus of claim 1, wherein at least one layer of the one or more layers comprises an inductive power coil configured to receive power for operating the one or more electronic devices.

3. The apparatus of claim 2, wherein at least one layer of the one or more layers comprises one or more light emitting diodes (LEDs), wherein the light emitting diodes (LEDs) are configured to receive power from the inductive power coil.

4. The apparatus of claim 3, wherein at least one layer of the one or more layers comprises a wireless module, wherein the wireless module is coupled to the inductive power coil and to the light emitting diodes (LEDs), and wherein the wireless module is configured to receive commands to operate the light emitting didoes (LEDs) and to operate the light emitting diodes (LEDs) based, at least in part, on the received commands.

5. The apparatus of claim 1, wherein the apparatus further comprises an adhesive backing on the flexible substrate to form a sticker.

6. The apparatus of claim 1, further comprising a flexible battery coupled to the one or more layers.

7. The apparatus of claim 1, wherein the flexible substrate is packaged using polymer on cellulose manufacturing using 3D printing.

8. The apparatus of claim 2, wherein at least one layer of the one or more layers comprises one or more sensors, wherein the one or more sensors are configured to receive power from the inductive power coil, wherein at least one layer of the one or more layers comprises a wireless module, wherein the wireless module is coupled to the inductive power coil and to the one or more sensors, and wherein the wireless module is configured to receive data from the one or more sensors and to wirelessly communicate the received data to a client device.

9. The apparatus of claim 8, wherein the one or more sensors comprises at least one of a smoke detector and a carbon monoxide detector.

10. The apparatus of claim 8, wherein the one or more sensors comprises a rain gauge.

11. A method, comprising:
providing a flexible substrate comprising a base for one or more electronic devices; and
providing one or more layers on the flexible substrate, wherein the one or more layers form components for the one or more electronic devices,
wherein a top layer of the one or more layers is exposed to an environment around the top layer and includes components to interact with the environment.

12. The method of claim 11, wherein at least one layer of the one or more layers comprises an inductive power coil configured to receive power for operating the one or more electronic devices.

13. The method of claim 12, wherein at least one layer of the one or more layers comprises one or more light emitting diodes (LEDs), wherein the light emitting diodes (LEDs) are configured to receive power from the inductive power coil.

14. The method of claim 13, wherein at least one layer of the one or more layers comprises a wireless module, wherein the wireless module is coupled to the inductive power coil and to the light emitting diodes (LEDs), and wherein the wireless module is configured to receive commands to operate the light emitting didoes (LEDs) and to operate the light emitting diodes (LEDs) based, at least in part, on the received commands.

15. The method of claim 11, further comprising providing an adhesive backing on the flexible substrate to form a sticker.

16. The method of claim 11, further comprising coupling a flexible battery to the one or more layers.

17. The method of claim 11, further comprising packaging the flexible substrate using polymer on cellulose manufacturing using 3D printing.

18. The method of claim 12, wherein at least one layer of the one or more layers comprises one or more sensors, wherein the one or more sensors are configured to receive power from the inductive power coil, wherein at least one layer of the one or more layers comprises a wireless module, wherein the wireless module is coupled to the inductive power coil and to the one or more sensors, and wherein the wireless module is configured to receive data from the one or more sensors and to wirelessly communicate the received data to a client device.

19. The method of claim 18, wherein the one or more sensors comprises at least one of a smoke detector and a carbon monoxide detector.

20. The method of claim 18, wherein the one or more sensors comprises a rain gauge.
